(19) United States
(12) Patent Application Publication
(10) Pub. No.: US 2006/0223857 A1
(43) Pub. Date: Oct. 5, 2006

(54) Combination of a serotonin reuptake inhibitor and a glycine transporter type 1 inhibitor for the treatment of depression

(76) Inventors: Michael Didriksen, (US); Sandra Willigers, Bagsvaerd (DK); Jørn Arnt, Solrød Strand (DK)

Correspondence Address: DARBY & DARBY P.C., P. O. BOX 5257, NEW YORK, NY, 10150-5257, US

(21) Appl. No.: 10/568,133

(22) PCT Filed: Aug. 18, 2004
(86) PCT No.: PCT/DK04/00547

§ 371 Date: May 9, 2006

Related U.S. Application Data
(60) Provisional application No. 60/496,738, filed on Aug. 21, 2003.

(30) Foreign Application Priority Data
Aug. 21, 2003 (DK) PA200301198

(51) Int. Cl. A61K 31/445; A61K 31/381; A61K 31/343; A61K 31/137

(52) U.S. Cl. 514/317; 514/469; 514/649; 514/447

(57) ABSTRACT

The present invention relates to the use of a compound, which is a serotonin reuptake inhibitor, and a compound, which is a GlyT-1 inhibitor for the preparation of a pharmaceutical composition for the treatment of depression, anxiety disorders and other affective disorders. In particular the present invention relates to treatment of depression, anxiety disorders and other affective disorders, such as generalized anxiety disorder, panic anxiety, obsessive compulsive disorder, acute stress disorder, post traumatic stress disorder and social anxiety disorder, eating disorders such as bulimia, anorexia and obesity, phobias, dysthymia, premenstrual syndrome, cognitive disorders, impulse control disorders, attention deficit hyperactivity disorder, drug abuse or any other disorder responsive to serotonin reuptake inhibitors. The present invention also relates to a pharmaceutical composition comprising a serotonin reuptake inhibitor, and a GlyT-1 inhibitor.

The present invention relates to the combination of a serotonin reuptake inhibitor (SRI) and a glycine transporter type 1 (GlyT-1) inhibitor. Accordingly, the present invention relates to the use of certain compounds, and to compositions of compounds having serotonin reuptake inhibiting activity and GlyT-1 inhibitor activity for the treatment of depression and other affective disorders.

### BACKGROUND

Selective serotonin reuptake inhibitors (hereinafter referred to as SSRIs) have become first choice therapeutics in the treatment of depression, certain forms of anxiety and social phobias, because they are effective, well tolerated and have a favourable safety profile compared to the classic tricyclic antidepressants.

However, clinical studies on depression and anxiety disorders indicate that non-response to SSRIs is substantial, up to 30%. Another, often neglected, factor in antidepressant treatment is compliance, which has a rather profound effect on the patient's motivation to continue pharmacotherapy.

First of all, there is the delay in therapeutic effect of SSRIs. Sometimes symptoms even worsen during the first weeks of treatment. Secondly, sexual dysfunction is a side effect common to all SSRIs. Without addressing these problems, real progress in the pharmacotherapy of depression and anxiety disorders is not likely to happen.

In order to cope with non-response, psychiatrists sometimes make use of augmentation strategies. Augmentation of antidepressant therapy may be accomplished through the co-administration of mood stabilizers such as lithium carbonate or triiodothyronin or by the use of electroshock.

In 1993, an augmentation strategy with pindolol was described by Artigas et al. in Trends Pharmacol Sci. 1993, 14, p 262-263. Artigas' idea is based on intracerebral microdialysis experiments in animals. In fact, later neurochemical studies built on the desensitization hypothesis by Blier and co-workers stated that the delay in therapeutic effect of antidepressants is related to a gradual desensitization of 5-HT autoreceptors (Blier et al. J. Clin. Psycipharmacol. 1987, 7 suppl. 6, 24S-35S). A key point in their hypothesis is that the effects of SSRIs on the release-controlling somatodendritic autoreceptors (5-HT_{1A}) limit the release of 5-HT in terminal areas and thus the effect of 5-HT uptake inhibition in those regions. This is supported by microdialysis experiments in rats, showing that the increase in extracellular 5-HT elicited by a single dose of an SSRI is augmented by co-administration of a 5-HT_{1A} autoreceptor antagonist (Invernizzi et al. Brain Res, 1992, 584, p 322-324 and Hjorth, S., J. Neurochem, 1993, 60, p 776-779).

The effect of combined administration of a compound that inhibits serotonin reuptake and a 5-HT_{1A} receptor antagonist has been evaluated in several studies (Innis, R. B. et al. Eur. J. Pharmacol 1987, 143, p. 1095-204 and Gartside, S. E., Br. J. Pharmacol, 1995, 115, p 1064-1070, Blier, P. et al. Trends in Pharmacol. Science 1994, 15, 220). In these studies it was found that 5-HT_{1A} receptor antagonists would abolish the initial brake on 5-HT neurotransmission induced by the serotonin reuptake inhibitors and thus produce an immediate boost of 5-HT transmission and a rapid onset of therapeutic action.

Several patent applications have been filed which cover the use of a combination of a 5-HT_{1A} antagonist and a serotonin reuptake inhibitor for the treatment of depression (see e.g. EP-A2-687 472 and EP-A2-714 663).

Another approach to increase terminal 5-HT would be through blockade of the 5-HT_{1 B} autoreceptor. Microdialysis experiments in rats have indeed shown that increase of hippocampal 5-HT by citalopram is potentiated by GMC 2-29, an experimental 5-HT_{1B} receptor antagonist.

Several patent applications covering the combination of an SSRI and a 5-HT_{1B} antagonist or partial agonist have also been filed (WO 97/28141, WO 96/03400, EP-A-701819 and WO 99/13877).

Glutamate is the most important excitatory neurotransmitter in the brain mediating its effect via ionotropic and metabotropic receptors. Inonotropic NMDA receptors are involved in the glutamatergic excitation of GABAergic, serotonergic, dopaminergic, and adrenergic neurons.

The NMDA receptor is positive modulated by glycine. Functional NMDA receptor complexes are formed by combinations of NR1 and NR2 subunits, which contain the glycine and glutamate recognition sites, respectively (Danysz W & Parsons C. G., Pharmacological reviews, vol 50: pp 597-664 (1998)).

GlyT-1 transporters located in the adjacent glia cells regulate the endogenous level of glycine in the vicinity of the NMDA receptor complex. Consequently, inhibiting the GlyT-1 transporter results in increased level of glycine and NMDA receptor activation (Danysz W & Parsons C. G., Pharmacological reviews, vol 50: pp 597-664 (1998)).

In preclinical models of depression (Chronic severe stress and Chronic mild stress) the involvement of the NMDA receptor complex has been shown (Novak G. et al., Polish Journal of Pharmacology, vol 58: pp 365-369 (1998)). Further, Glycine site partial agonists show antidepressant like effect in the Chronic mild stress model (Papp M. & Moryl E., European Journal of Pharmacology, vol 316: pp 145-151 (1996))

### DESCRIPTION OF THE INVENTION

It has now surprisingly been found that a GlyT-1 inhibitor will augment the effect of an SRI, in particular an SSRI on extracellular 5-HT levels.

It is therefore suggested that the combination of an SSRI and a GlyT-1 inhibitor, provide 5-HT reuptake inhibitory and GlyT-1 inhibitor properties, and would have a better efficacy and faster onset than an SSRI alone.

The present invention thus provides:

The use of a GlyT-1 inhibitor for the preparation of a pharmaceutical composition to be used in combination with a serotonin reuptake inhibitor (SRI).

The present invention relates to the use of a compound, which is a serotonin reuptake inhibitor, and another compound, which is a GlyT-1 inhibitor for the preparation of a pharmaceutical composition for the treatment of depression, anxiety disorders and other affective disorders, such as generalized anxiety disorder, panic anxiety, obsessive compulsive disorder, acute stress disorder, post traumatic stress disorder and social anxiety disorder, eating disorders such as bulimia, anorexia and obesity, phobias, dysthymia, premenstrual syndrome, cognitive disorders, impulse control disorders, attention deficit hyperactivity disorder, drug abuse or any other disorder responsive to serotonin reuptake inhibitors.

The present invention also relates to the use of a GlyT-1 inhibitor for the preparation of a pharmaceutical composition useful for augmenting and providing faster onset of the therapeutic effect of a serotonin reuptake inhibitor. Moreover, the present invention also relates to the use of a GlyT-1 inhibitor for the preparation of a pharmaceutical composition useful for augmenting or providing faster onset of the therapeutic effect of a serotonin reuptake inhibitor.

Moreover the invention relates to the use of a combination of a compound, which is a serotonin reuptake inhibitor, and a compound, which is a GlyT-1 inhibitor, for the preparation of a pharmaceutical composition or kit-of-parts (kit) useful for the treatment of depression, anxiety disorders and other affective disorders, such as generalized anxiety disorder, panic anxiety, obsessive compulsive disorder, acute stress disorder, post traumatic stress disorder and social anxiety disorder, eating disorders such as bulimia, anorexia and obesity, phobias, dysthymia, premenstrual syndrome, cognitive disorders, impulse control disorders, attention deficit hyperactivity disorder, drug abuse or any other disorder responsive to serotonin reuptake inhibitors.

Furthermore the invention relates to the use of a compound, which is a serotonin reuptake inhibitor, and a compound, which is a GlyT-1 inhibitor, for the preparation of a kit for use in the treatment of depression, anxiety disorders and other affective disorders, such as generalized anxiety disorder, panic anxiety, obsessive compulsive disorder, acute stress disorder, post traumatic stress disorder and social anxiety disorder, eating disorders such as bulimia, anorexia and obesity, phobias, dysthymia, premenstrual syndrome, cognitive disorders, impulse control disorders, attention deficit hyperactivity disorder, drug abuse or any other disorder responsive to serotonin reuptake inhibitors.

In a further aspect the invention relates to a pharmaceutical composition comprising a combination of a compound, which is a serotonin reuptake inhibitor, and another compound, which is a GlyT-1 inhibitor, and optionally pharmaceutically acceptable carriers or diluents.

In a further aspect the invention relates to a kit comprising a combination of a compound, which is a serotonin reuptake inhibitor, and another compound, which is a GlyT-1 inhibitor, and optionally pharmaceutically acceptable carriers or diluents.

In yet another aspect the invention relates to a method for the treatment of depression, anxiety disorders and other affective disorders, such as generalized anxiety disorder, panic anxiety, obsessive compulsive disorder, acute stress disorder, post traumatic stress disorder and social anxiety disorder, eating disorders such as bulimia, anorexia and obesity, phobias, dysthymia, premenstrual syndrome, cognitive disorders, impulse control disorders, attention deficit hyperactivity disorder, drug abuse or any other disorder responsive to serotonin reuptake inhibitors comprising administering to a person in need thereof a therapeutically effective amount of a combination of a compound, which is a serotonin reuptake inhibitor and a compound, which is a GlyT-1 inhibitor.

In a further aspect, the invention relates to a method for the treatment of depression, anxiety disorders and other affective disorders, such as generalized anxiety disorder, panic anxiety, obsessive compulsive disorder, acute stress disorder, post traumatic stress disorder and social anxiety disorder, eating disorders such as bulimia, anorexia and obesity, phobias, dysthymia, premenstrual syndrome, cognitive disorders, impulse control disorders, attention deficit hyperactivity disorder, drug abuse or any other disorder responsive to serotonin reuptake inhibitors comprising administering a compound, which is a GlyT-1 inhibitor and a compound, which is a serotonin reuptake inhibitor, or a compound which causes an elevation in the level extracellular serotonin, to an individual in need thereof.

In a further aspect, the invention relates to a method for augmenting and/or providing faster onset of the therapeutic effect of a serotonin reuptake inhibitor, or any other compound which causes an elevation in the level of extracellular serotonin, comprising administering a GlyT-1 inhibitor to an individual to be treated with or undergoing treatment with the serotonin reuptake inhibitor, or any other compound which causes an elevation in the level of extracellular serotonin. Such individual is preferably a human, such as male or female human, child, adult or elderly.

Each of the medical indications: depression, anxiety disorders and other affective disorders, including generalized anxiety disorder, panic anxiety, obsessive compulsive disorder, acute stress disorder, post traumatic stress disorder or social anxiety disorder, eating disorders such as bulimia, anorexia and obesity, phobias, dysthymia, premenstrual syndrome, cognitive disorders, impulse control disorders, attention deficit hyperactivity disorder, drug abuse and any other disorder responsive to a SRI is intended to be an individual embodiment. Accordingly, whenever mentioned in the present description, each of the indications specified above may be claimed individually.

Whenever the indications depression, anxiety disorders and other affective disorders, including generalized anxiety disorder, panic anxiety, obsessive compulsive disorder, acute stress disorder, post traumatic stress disorder or social anxiety disorder, eating disorders such as bulimia, anorexia and obesity, phobias, dysthymia, premenstrual syndrome, cognitive disorders, impulse control disorders, attention deficit hyperactivity disorder, drug abuse and any other disorder responsive to a SRI are mentioned in relation to use of a GlyT-1 inhibitor and a SRI, a pharmaceutical composition, a kit, a method of treatment and a method for the identification of compounds useful for treatment each indication is intended to be an individual embodiment. Accordingly, each of the indications specified above may individually be claimed together with said use of a GlyT-1 inhibitor and an SRI, pharmaceutical composition, kit, method of treatment and method for the identification of compounds useful for treatment.

In a particular embodiment, the SRI is a selective serotonin reuptake inhibitor (SSRI).

In another particular embodiment, a GlyT-1 inhibitor, which is selective for the glycine transporter type 1 is used according to the invention.

The pharmaceutical composition or kit according to the invention may be administered by simultaneous administration. The term “simultaneous administration” as used herein means, that the GlyT-1 inhibitor and the SRI are administered with a time separation of no more than 15 minutes, such as at most 10 minutes, such as at most 5 minutes or such as at most 2 minutes. The GlyT-1 inhibitor and the SRI may be contained in the “same unit dosage form” or in “discrete dosage forms”. As used herein, the term “same unit dosage form” means a dosage form comprising both the SRI and the GlyT-1 inhibitor. As used herein, the term “discrete dosage form” means that the GlyT-1 inhibitor is comprised in one dosage form and that the SRI is comprised in another dosage form.

Simultaneous administration of the GlyT-1 inhibitor and the SRI is optionally combined with administration of supplementary doses of GlyT-1 inhibitor. The supplementary doses of GlyT-1 inhibitor may be given for instance 1, 2, 3 or 4 times a day whereas the SRI and the GlyT-1 inhibitor which are administered by “simultaneous administration” may be given one or more times a day, e.g. once daily or e.g. twice daily. Accordingly:

a) the GlyT-1 inhibitor and the SRI may be administered by simultaneous administration once daily and supplementary doses of GlyT-1 inhibitor may be administered 1, 2, 3 or 4 times a day, such as 1, 2 or 3 times a day, such as once or twice daily, such as twice daily or such as once daily, or

b) the GlyT-1 inhibitor and the SRI may be administered by simultaneous administration twice daily and supplementary doses of GlyT-1 inhibitor may be administered 1, 2, 3 or 4 times a day, such as 1, 2 or 3 times a day, such as once or twice daily, such as twice daily or such as once daily.

Alternatively, the pharmaceutical composition or kit according to the invention is administered by sequential administration. The term “sequential administration” as used herein means that one (1) or more daily doses of the GlyT-1 inhibitor and 1 or more daily doses of SRI are administered with a time separation between two administered doses of more than 15 minutes and less than 4 hours, such as more than 2 hours and less than 4 hours, such as more than 15 minutes and less than 2 hours, such as more than 1 hour and less than 2 hours, such as more than 30 minutes and less than 1 hour, such as more than 15 minutes and less than 30 minutes. Either the SRI or the GlyT-1 inhibitor may be administered first. The GlyT-1 inhibitor and the SRI are contained in discrete dosage forms, optionally contained in the same container or package. Typically, 1, 2, 3, 4 or 5 daily doses of GlyT-1 inhibitor and 1 or 2 daily doses of SRI may be administered. Accordingly:

a) the GlyT-1 inhibitor and the SRI may be administered once daily and the GlyT-1 inhibitor may be administered 1, 2, 3, 4 or 5 times a day, such as 1, 2, 3 or 4 times a day, such as 1, 2 or 3 times a day, such as once or twice daily, such as twice daily or such as once daily, or

b) the GlyT-1 inhibitor and the SRI may be administered twice daily and the GlyT-1 inhibitor may be administered 1, 2, 3, 4 or 5 times a day, such as 1, 2, 3 or 4 times a day, such as 1, 2 or 3 times a day, such as once or twice daily, such as twice daily or such as once daily.

Accordingly, the pharmaceutical composition or kit according to the invention may be adapted for simultaneous administration of the active ingredients, or it may be adapted for sequential administration of the active ingredients. When the pharmaceutical composition or kit is adapted for simultaneous administration, the active ingredients may be contained in the same unit dosage form. When the pharmaceutical composition or kit is adapted for sequential administration, the active ingredients are contained in discrete dosage forms, optionally contained in the same container or package. As used herein, an “active ingredient” means a SRI or a GlyT-1 inhibitor.

A kit comprises a preparation of the GlyT-1 inhibitor in a first-unit dosage form, and the SRI in a second-unit dosage form, and container means for containing said first and second dosage forms.

In a further embodiment, the GlyT-1 inhibitor is selected from any one of the compounds disclosed in WO0208216, such as any one of
N-{3-[5-Cyano-1-(4-fluorophenyl)-1,3-dihydroisobenzofuran-1-yl]-1-propyl}glycine ethyl ester,
N-{3-[5-Cyano-1-(4-fluorophenyl)-1,3-dihydroisobenzofuran-1-yl]-1-propyl}-N-methylglycine ethyl ester,
N-{3-[5-Cyano-1-(4-fluorophenyl)-1,3-dihydroisobenzofuran-1-yl]-1-propyl}glycine,
N-{3-[5-Cyano-1-(4-fluorophenyl)-1,3-dihydroisobenzofuran-1-yl]-1-propyl}-N-methylglycine,
N-{3-[1-(3-chlorophenyl)-1,3-dihydroisobenzofuran-1-yl]-1-propyl}-N-methylglycine,
N-{3-[1-(3-trifluoromethylphenyl)-1,3-dihydroisobenzofuran-1-yl]-1-propyl}-N-methylglycine,
N-{3-[1-(3-trifluoromethylphenyl)-1,3-dihydroisobenzofuran-1-yl]-1-propyl}-N-methyl(1-ethyl)glycine,
N-{3-[1-(4-methylphenyl)-1,3-dihydroisobenzofuran-1-yl]-1-propyl}-N-methylglycine,
N-{3-[1-(4-fluorophenyl)-1,3-dihydroisobenzofuran-1-yl]-1-propyl}-N-methylglycine,
N-{3-[1-(4-fluorophenyl)-1,3-dihydroisobenzofuran-1-yl]-1-propyl}-N-methylglycine,
N-{3-[1-(4-fluorophenyl)-1,3-dihydroisobenzofuran-1-yl]-1-propyl}-N-methyl(1-ethyl)glycine,
N-{3-[4-chloro-1-(3-methyl-4-fluorophenyl)-1,3-dihydroisobenzofuran-1-yl]-1-propyl}-N-methylglycine,
N-{3-[4-chloro-1-(4-chlorophenyl)-1,3-dihydroisobenzofuran-1-yl]-1-propyl}-N-methylglycine,
N-{3-[5-chloro-1-(4-chlorophenyl)-1,3-dihydroisobenzofuran-1-yl]-1-propyl}-N-methylglycine,
N-{3-[6-chloro-1-(3-methyl-4-fluorophenyl)-1,3-dihydroisobenzofuran-1-yl]-1-propyl}-N-methylglycine,
N-{3-[6-chloro-1-(4-chlorophenyl)-1,3-dihydroisobenzofuran-1-yl]-1-propyl}-N-methylglycine,
N-{3-[6-chloro-1-(4-methylphenyl)-1,3-dihydroisobenzofuran-1-yl]-1-propyl}-N-methylglycine,
N-{3-[6-chloro-1-(4-methoxyphenyl)-1,3-dihydroisobenzofuran-1-yl]-1-propyl}-N-methylglycine,
N-{3-[5-fluoro-1-(4-chlorophenyl)-1,3-dihydroisobenzofuran-1-yl]-1-propyl}-N-methylglycine,
N-{3-[5-fluoro-1-(4-methoxyphenyl)-1,3-dihydroisobenzofuran-1-yl]-1-propyl}-N-methylglycine,
N-{3-[5-trifluoromethyl-1-(4-fluorophenyl)-1,3-dihydroisobenzofuran-1-yl]-1-propyl}-N-methylglycine,
N-{3-[5-trifluoromethyl-1-(4-fluorophenyl)-1,3-dihydroisobenzofuran-1-yl]-1-propyl}-N-methylalanine,
N-{3-[5-cyano-1-(3-methyl-4-fluorophenyl)-1,3-dihydroisobenzofuran-1-yl]-1-propyl}-N-methylglycine,
N-{3-[5-cyano-1-(4-cyanophenyl)-1,3-dihydroisobenzofuran-1-yl]-1-propyl}-N-methylalanine,
N-{3-[5-cyano-1-(4-methoxyphenyl)-1,3-dihydroisobenzofuran-1-yl]-1-propyl}-N-methylglycine,
N-{3-[5-cyano-1-(4-fluorophenyl)-1,3-dihydroisobenzofuran-1-yl]-1-propyl}-N-methylglycine, N-{2-[5-cyano-1-(4-fluorophenyl)-1,3-dihydroisobenzofuran-1-yl]ethyl}-N-methylglycine,
N-{3-[5-Chloro-1-(4-chloro-phenyl)-indan-1-yl]-propyl}-N-methylglycine,
N-{3-[5-Chloro-1-(4-chloro-phenyl)-indan-1-yl]-propyl}-N-methylalanine,
N-{3-[3-cyclo-1-(4-methylphenyl)-1,3-dihydroisobenzofuran-1-yl]-1-propyl}-N-methylglycine,
N-[3-(3,3-Dimethyl-1-phenyl-1,3-dihydro-benzo[c]thiophen-1-yl)-propyl]-N-methylglycine,
N-[3-(3,3-Dimethyl-1-phenyl-1,3-dihydro-benzo[c]thiophen-1-yl)-propyl]-N-methylglycine,
N-{3-[1-(4-Fluoro-phenyl)-3,3-dimethyl-1,3-dihydro-isobenzofuran-1-yl]-propyl}-N-methylglycine,
N-{3-[5-Bromo-1-(4-chlorophenyl)-1,3-dihydroisobenzofuran-1-yl]-1-propyl}-N-methylglycine,
N-{2-[1-(4-Chloro-phenyl)-3,3-dimethyl-1,3-dihydro-isobenzofuran-1-yl]-ethyl}-N-methylglycine,
N-[3-(3-methyl-1-phenyl-1H-inden-1-yl)-propyl]-N-methylglycine,
N-[3-(5-Chloro-1-thiophen-2-yl-1,3-dihydro-isobenzofuran-1-yl)-propyl]-N-methylglycine,
N-[3-(5-Chloro-1-thiophen-2-yl-1,3-dihydro-isobenzofuran-1-yl)-propyl]-N-methyl(1-ethyl)-glycine,
N-[3-(3-methyl-1-phenyl-1,3-dihydro-isobenzofuran-1-yl)-propyl]-N-methylalanine,
N-[3-(3-methyl-1-phenyl-1,3-dihydro-isobenzofuran-1-yl)-propyl]-N-methyl(1-ethyl)-glycine,
N-[3-(3,3-Dimethyl-1-phenyl-1,3-dihydro-isobenzofuran-1-yl)-ethyl]-N-methylalanine,
N-[3-(3,3-Dimethyl-1-(4-fluoro-phenyl)-1,3-dihydro-isobenzofuran-1-yl)-ethyl]-N-methylalanine,
N-[3-(3,3-Dimethyl-1-phenyl-1,3-dihydro-isobenzofuran-1-yl)-ethyl]-N-methyl-(1-ethyl)glycine,
N-[3-(3,3-Dimethyl-1-(4-fluoro-phenyl)-1,3-dihydro-isobenzofuran-1-yl)-ethyl]-N-methyl-(1-ethyl)glycine,
N-[3-(3,3-Diethyl-1-phenyl-1,3-dihydro-isobenzofuran-1-yl)-propyl]-N-methylalanine,
N-[3-(3,3-Diethyl-1-(4-chloro-phenyl)-1,3-dihydro-isobenzofuran-1-yl)-propyl]-N-methylalanine,
N-[3-(3,3-Diethyl-1-(4-chloro-phenyl)-1,3-dihydro-isobenzofuran-1-yl)-propyl]-N-methylglycine,
N-[3-(1-phenyl-1,3-dihydro-benzo[c]thiophen-1-yl)-propyl]-N-methylalanine,
N-{3-[1-(4-Chloro-phenyl)-3,3-dimethyl-indan-1-yl]-propyl}-N-methylglycine,
N-{3-[1-(4-Chloro-phenyl)-3,3-diethyl-1,3-dihydro-isobenzofuran-1-yl]-propyl}-N-methyl-alanine,
N-[2-(3-methyl-1-phenyl-indan-1-yl)-ethyl]-amino}-N-methyl alanine,
N-[3-(1-phenyl-(1H)-inden-1-yl)-propyl]-N-methyl-alanine,
N-{3-[1-(4-Fluoro-phenyl)-5-(4-trifluoromethyl-phenyl)-1,3-dihydro-isobenzofuran-1-yl]-propyl}-N-methyl-glycine,
N-{3-[5-Chloro-1-(4-chloro-phenyl)-indan-1-yl]-propyl}-N-methyl-glycine,
N-{3-[5-Chloro-1-(4-chloro-phenyl)-indan-1-yl]-propyl}-N-methyl-alanine,
N-{3-[1-(4-chloro-phenyl)-5-(4-trifluoromethyl-phenyl)-1,3-dihydro-isobenzofuran-1-yl]-ethyl}-N-methyl-glycine,
N-{3-[1-(4-Chloro-phenyl)-5-(4-methyl-phenyl)-1,3-dihydro-isobenzofuran-1-yl]-ethyl}-N-methyl-glycine,
N-{3-[1-(4-Chloro-phenyl)-5-(4-methoxy-phenyl)-1,3-dihydro-isobenzofuran-1-yl]-ethyl}-N-methyl-glycine,
N-{3-[1-(4-Chloro-phenyl)-5-(2-thiophenyl)-1,3-dihydro-isobenzofuran-1-yl]-ethyl}-N-methyl-glycine,
N-{3-[1-(4-Chloro-phenyl)-5-(4-methyl-phenyl)-1,3-dihydro-isobenzofuran-1-yl]-propyl}-N-methyl-glycine,
N-{3-[1-(4-Chloro-phenyl)-5-(4-methoxy-phenyl)-1,3-dihydro-isobenzofuran-1-yl]-propyl}-N-methyl-glycine,
N-{3-[1-(4-chloro-phenyl)-5-(4-trifluoromethyl-phenyl)-1,3-dihydro-isobenzofuran-1-yl]-propyl}-N-methyl-glycine,
N-{3-[1-(4-Chloro-phenyl)-5-(4-chloro-phenyl)-1,3-dihydro-isobenzofuran-1-yl]-ethyl}-N-methyl-glycine,
N-{2-[1-(4-Chloro-phenyl)-5-(5-chloro-thiophen-2-yl)-1,3-dihydro-isobenzofuran-1-yl]-ethyl}-N-methyl-glycine,
N-{3-[1-(4-Chloro-phenyl)-5-(3-methyl-phenyl)-1,3-dihydro-isobenzofuran-1-yl]-ethyl}-N-methyl-glycine,
N-{3-[1-(4-Chloro-phenyl)-5-(2-methyl-phenyl)-1,3-dihydro-isobenzofuran-1-yl]-ethyl}-N-methyl-glycine,
N-{3-[1-(4-Chloro-phenyl)-5-(2,5-dichloro-phenyl)-1,3-dihydro-isobenzofuran-1-yl]-ethyl}-N-methyl-glycine,
N-{3-[1-(4-chloro-phenyl)-5-(3-trifluoromethyl-phenyl)-1,3-dihydro-isobenzofuran-1-yl]-ethyl}-N-methyl-glycine,
N-{3-[1-(4-chloro-phenyl)-5-(3-trifluoromethyl-phenyl)-1,3-dihydro-isobenzofuran-1-yl]-propyl}-N-methyl-glycine,
N-{3-[1-(4-Chloro-phenyl)-5-(3,4-dichloro-phenyl)-1,3-dihydro-isobenzofuran-1-yl]-ethyl}-N-methyl-glycine,
N-{3-[1-(4-Chloro-phenyl)-5-(4-chloro-phenyl)-1,3-dihydro-isobenzofuran-1-yl]-propyl}-N-methyl-glycine,
N-{3-[1-(4-Chloro-phenyl)-5-(3-methyl-phenyl)-1,3-dihydro-isobenzofuran-1-yl]-propyl}-N-methyl-glycine,
N-{3-[1-(4-Chloro-phenyl)-5-(2-methyl-phenyl)-1,3-dihydro-isobenzofuran-1-yl]-propyl}-N-methyl-glycine,
N-{3-[1-(4-Chloro-phenyl)-5-(2,5-dichloro-phenyl)-1,3-dihydro-isobenzofuran-1-yl]-propyl}-N-methyl-glycine,
N-{3-[1-(4-Chloro-phenyl)-5-(3,4-dichloro-phenyl)-1,3-dihydro-isobenzofuran-1-yl]-propyl}-N-methyl-glycine,
N-{3-[1-(4-chloro-phenyl)-5-(2-trifluoromethyl-phenyl)-1,3-dihydro-isobenzofuran-1-yl]-propyl}-N-methyl-glycine,
   or a pharmaceutically acceptable addition salt thereof.

In a further embodiment, the GlyT-1 inhibitor is selected from any one of the compounds disclosed in WO03/053942, such as any one of
(±)-{4-[2-(4-Methoxy-phenylsulfanyl)-phenyl]-trans-2,5-dimethyl-piperazin-1-yl}-acetic acid,
(±)-{4-[2-(4-Chloro-phenylsulfanyl)-phenyl]-trans-2,5-dimethyl-piperazin-1-yl}-acetic acid,
(±)-{4-[2-(4-tert-Butyl-phenylsulfanyl)-phenyl]-trans-2,5-dimethyl-piperazin-1-yl}-acetic acid,
(±)-{4-[2-(4-Fluoro-phenylsulfanyl)-phenyl]-trans-2,5-dimethyl-piperazin-1-yl}-acetic acid,
(±)-{4-[2-(4-tert-Butyl-phenylsulfanyl)-phenyl]-2-methyl-piperazin-1-yl}-acetic acid,
(±)-{4-[2-(4-iso-Propyl-phenylsulfanyl)-phenyl]-2-methyl-piperazin-1-yl}-acetic acid,
(±)-2-{4-[2-(4-tert-Butyl-phenylsulfanyl)-phenyl]-trans-2,5-dimethylpiperazin-1-yl}-propionic acid,
{4-[5-Chloro-2-(4-methoxy-phenylsulfanyl)-phenyl]-2(R)-methyl-piperazin-1-yl}-acetic acid,
{4-[2-(4-Methoxy-phenylsulfanyl)-phenyl]-2(R),5(S)-dimethyl-piperazin-1-yl}-acetic acid,
{4-[5-Chloro-2-(4-methoxy-phenylsulfanyl)-phenyl]-2,2-dimethyl-piperazin-1-yl}-acetic acid,
(±)-{4-[5-Chloro-2-(4-trifluoromethyl-phenylsulfanyl)-phenyl]-2-methyl-piperazin-1-yl}-acetic acid,
{4-[5-Chloro-2-(3-methoxy-phenylsulfanyl)-phenyl]-2(R)-methyl-piperazin-1-yl}-acetic acid,
(±)-{4-[2-(4-Phenyl-phenyloxy)-phenyl]-2-methyl-piperazin-1-yl}-acetic acid,
(±)-{4-[2-(4-Methyl-phenylsulfanyl)-phenyl]-trans-2,5-dimethyl-piperazin-1-yl}-acetic acid,
(±)-{4-[2-(4-iso-Propyl-phenylsulfanyl)-phenyl]-trans-2,5-dimethyl-piperazin-1-yl}-acetic acid,
(±)-{4-[2-(2,4-Dimethyl-phenylsulfanyl)-phenyl]-trans-2,5-dimethyl-piperazin-1-yl}-acetic acid,
(±)-2-{4-[2-(4-tert-Butyl-phenylsulfanyl)-phenyl]-3-methylpiperazin-1-yl}-propionic acid,
{4-[2-(4-Isopropyl-phenylsulfanyl)-phenyl]-piperazin-1-yl}-acetic acid,
(±)-2-{4-[2-(4-Methoxy-phenylsulfanyl)-phenyl]-3-methyl-piperazin-1-yl}-propionic acid,
   or a pharmaceutically acceptable acid addition salt thereof.

Typical GlyT-1 inhibitors show inhibition below 20000 nM as IC₅₀ in the “[³H]-Glycine uptake” test described herein.

The invention also covers GlyT-1 inhibitors identified according to this method, but is not limited to these assay methods.

According to the invention, it has been found that co-administration of GlyT-1 inhibitors and a serotonin reuptake inhibitor produces a significant increase in the level of serotonin in terminal areas, as measured in microdialysis experiments, compared to the administration of the serotonin reuptake inhibitor alone.

According to the invention, animal studies have shown that GlyT-1 inhibitors may provide fast onset of therapeutic effect of serotonin reuptake inhibitors and potentiate the anxiolytic potential of serotonin reuptake inhibitors.

The use of a combination of a GlyT-1 inhibitor and a serotonin reuptake inhibitor may greatly reduce the amount of serotonin reuptake inhibitor necessary to treat depression and other affective disorders and may thus reduce the side effects caused by the serotonin reuptake inhibitor. In particular, the combination of a reduced amount of SRI and a GlyT-1 inhibitor may reduce the risk of SSRI-induced sexual dysfunction and sleep disturbances.

Co-administration of a GlyT-1 inhibitor and a serotonin reuptake inhibitor may also be useful for the treatment of refractory depression, i.e. depression, which cannot be treated appropriately by administration of a serotonin reuptake inhibitor alone. Typically, GlyT-1 inhibitors may be used as add-on therapy for the augmentation of the response to SRIs in patients where at least 40-60% reduction in symptoms has not been achieved during the first 6 weeks of treatment with an SRI.

Many antidepressants with serotonin reuptake inhibiting effect have been described in the literature. Any pharmacologically active compound, which primarily or partly exert its therapeutic effect via inhibition of serotonin reuptake in the CNS, may benefit from augmentation with a GlyT-1 inhibitor.

The following list contains a number of serotonin reuptake inhibitors, which may benefit from augmentation with a GlyT-1 inhibitor: citalopram, escitalopram, fluoxetine, R-fluoxetine, sertraline, paroxetine, fluvoxamine, venlafaxine, desmethylvenlafaxine, duloxetine, dapoxetine, vilazodone, nefazodone, imipramine, imipramine N-oxide, desipramine, pirandamine, dazepinil, nefopam, befuraline, fezolamine, femoxetine, clomipramine, cianoimipramine, litoxetine, cericlamine, seproxetine, WY 27587, WY 27866, imeldine, ifoxetine, indeloxazine, tiflucarbine, viqualine, milnacipran, bazinaprine, YM 922, S 33005, F 98214-TA, FI 4503, A 80426, EMD 86006, NS 2389, S33005, OPC 14523, alaproclate, cyanodothepine, trimipramine, quinupramine, dothiepin, amoxapine, nitroxazepine, McN 5652, McN 5707, VN 2222, L 792339, roxindole, YM 35992, Ol 77, Org 6582, Org 6997, Org 6906, amitriptyline, amitriptyline N-oxide, nortriptyline, CL 255.663, pirlindole, indatraline, LY 280253, LY 285974, LY 113.821, LY 214.281, CGP 6085 A, RU 25.591, napamezole, diclofensine, trazodone, BMY 42.569, NS 2389, sercloremine, nitroquipazine, ademethionine, sibutramine, desmethylsubitramine, didesmethylsubitramine, clovoxamine vilazodone. The compounds mentioned above may be used in the form of the base or a pharmaceutically acceptable acid addition salt thereof. Each of the serotonin reuptake inhibitors specified above is intended to be an individual embodiment. Accordingly, each of them and the use thereof may be claimed individually.

Compounds such as citalopram, escitalopram, fluoxetine, R-fluoxetine, sertraline, paroxetine, fluvoxamine, venlafaxine, desmethylvenlafaxine, duloxetine, dapoxetine, vilazodone, nefazodone, imipramine, imipramine N-oxide, desipramine, pirandamine, dazepinil, nefopam, befuraline, fezolamine, femoxetine, clomipramine, cianoimipramine, litoxetine, cericlamine, seproxetine, imeldine, ifoxetine, indeloxazine, tiflucarbine, viqualine, milnacipran, bazinaprine, alaproclate, cyanodothepine, trimipramine, quinupramine, dothiepin, amoxapine, nitroxazepine, roxindole, amitriptyline, amitriptyline N-oxide, nortriptyline, pirlindole, indatraline, napamezole, diclofensine, trazodone, sercloremine, nitroquipazine, ademethionine, sibutramine, desmethylsubitramine, didesmethylsubitramine, clovoxamine vilazodone,
N-[(1-[(6-Fluoro-2-naphthalenyl)methyl]-4-piperidinyl]amino]carbonyl]-3-pyridine carboxamide (WY 27587),
[trans-6-(2-chlorophenyl)-1,2,3,5,6,10b-hexahydropyrrolo-(2,1-a)isoquinoline] (McN 5707),
(dl-4-exo-amino-8-chloro-benzo-(b)-bicyclo[3.3.1]nona-2-6 alpha(10 alpha)-diene hydrochloride)(Org 6997),
(dl)-(5 alpha,8 alpha,9 alpha)-5,8,9,10-Tetrahydro-5,9-methanobenzocycloocten-8-amine hydrochloride (Org 6906),
-[2-[4-(6-fluoro-1H-indol-3-yl)-3,6-dihydro-1(2H)-pyridinyl]ethyl]-3-isopropyl-6-(methylsulphonyl)-3,4-dihydro-1H-2,1,3-benzothiadiazine-2,2-dioxide (LY393558), [4-(5,6-dimethyl-2-benzofuranyl)-piperidine] (CGP 6085),
dimethyl-[5-(4-nitro-phenoxy)-6,7,8,9-tetrahydro-5H-benzocyclohepten-7-yl]-amine (RU 25.591), 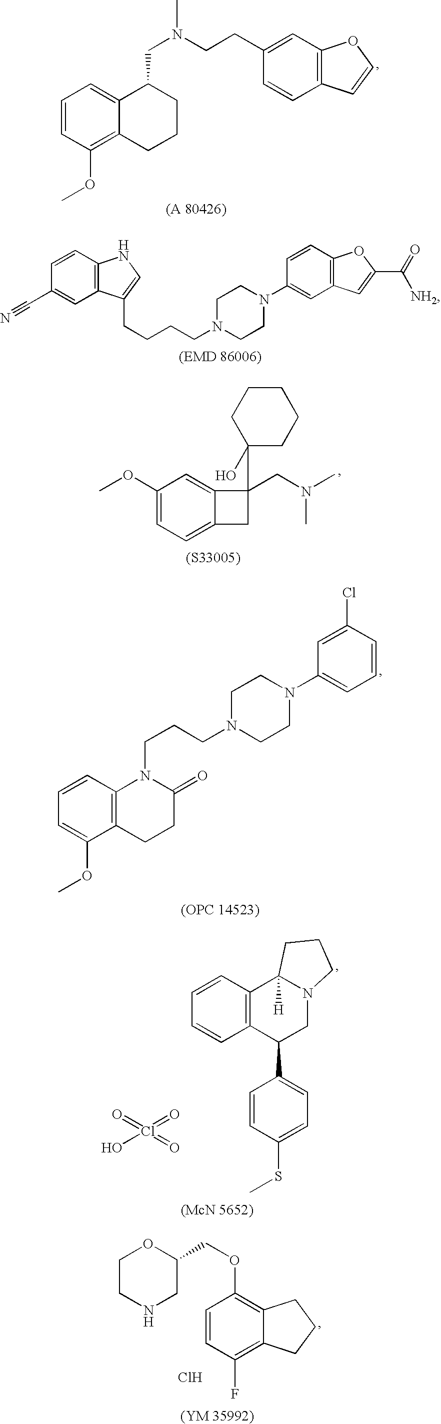 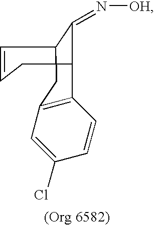
   are preferred. The compounds mentioned above may be used in the form of the base or a pharmaceutically acceptable acid addition salt thereof. Each of the serotonin reuptake inhibitors specified above is intended to be an individual embodiment. Accordingly, each of them and the use thereof may be claimed individually.

In a further embodiment, the SRI is selected from citalopram, escitalopram, fluoxetine, sertraline, paroxetine, fluvoxamine, venlafaxine, dapoxetine, vilazodone, duloxetine, nefazodone, imipramin, femoxetine and clomipramine, preferably citalopram, or escitalopram.

Typical serotonin reuptake inhibitors show serotonin reuptake inhibition below 10000 nM (IC₅₀) in the “Inhibition of the uptake of [³H]Serotonin into whole rat brain synaptosomes” test described herein.

Other therapeutic compounds, which may benefit from augmentation with GlyT-1 inhibitors, include compounds, which cause an elevation in the extracellular level of 5-HT in the synaptic cleft, although they are not serotonin reuptake inhibitors. One such compound is tianeptine.

Accordingly, other compounds than SRIs which cause an elevation in the extracellular level of serotonin, may be used instead of SRIs in every aspect of the invention as described herein.

The above list of serotonin reuptake inhibitors and other compounds, which cause an increase in the extracellular level of serotonin, may not be construed as limiting.

The term selective serotonin reuptake inhibitor (SSRI) means an inhibitor of the monoamine transporters, which has stronger inhibitory effect at the serotonin transporter than the dopamine and the noradrenaline transporters. Particularly preferred SSRIs according to the invention are citalopram, escitalopram, fluoxetine, fluvoxamine, sertraline, duloxetine, vilnazodone and paroxetine.

Pharmaceutical Compositions

Each of the active ingredients according to the invention may be administered alone or together or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the specific active ingredient or active ingredients chosen.

Pharmaceutical compositions for oral administration include solid dosage forms such as capsules, tablets, dragees, pills, lozenges, powders and granules. Where appropriate, they may be prepared with coatings such as enteric coatings or they may be formulated so as to provide controlled release of one or more active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

The pharmaceutical compositions of this invention or those which are manufactured in accordance with this invention may be administered by any suitable route, for example orally in the form of tablets, capsules, powders, syrups, etc., or parenterally in the form of solutions for injection. For preparing such compositions, methods well known in the art may be used, and any pharmaceutically acceptable carriers, diluents, excipients or other additives normally used in the art may be used.

A typical oral dosage of each of the active ingredients is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

For parenteral routes such as intravenous, intrathecal, intramuscular and similar administration, typically doses are in the order of about half the dose employed for oral administration.

The compounds of this invention are generally utilized as the free substance or as a pharmaceutically acceptable salt thereof. One example is a base addition salt of a compound having the utility of a free acid. When an active ingredient contains a free acid such salts are prepared in a conventional manner by treating a solution or suspension of a free acid of the active ingredient with a chemical equivalent of a pharmaceutically acceptable base.

For parenteral administration, solutions of one or more active ingredient in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Solutions for injections may be prepared by dissolving one or more active ingredients and possible additives in a part of the solvent for injection, preferably sterile water, adjusting the solution to a desired volume, sterilising the solution and filling it in suitable ampules or vials. Any suitable additive conventionally used in the art may be added, such as tonicity agents, preservatives, antioxidants, etc.

Suitable pharmaceutical carriers include inert solid diluents or fillers, sterile aqueous solution and various organic solvents.

Examples of solid carriers are lactose, terra alba, sucrose, cyclodextrin, talc, agar, pectin, acacia, stearic acid and lower alkyl ethers of cellulose corn starch, potato starch, talcum, magnesium stearate, gelatine, lactose, gums, and the like.

Any other adjuvants or additives usually used for such purposes such as colourings, flavourings, preservatives etc. may be used provided that they are compatible with the active ingredient or ingredients used.

Examples of liquid carriers are syrup, peanut oil, olive oil, phospho lipids, fatty acids, fatty acid amines, polyoxyethylene and water. Similarly, the carrier or diluent may include any sustained release material known in the art, such as glyceryl monostearate or glyceryl distearate, alone or mixed with a wax.

The pharmaceutical compositions formed by combining one or more active ingredients of the invention with the pharmaceutical acceptable carriers are then readily administered in a variety of dosage forms suitable for the disclosed routes of administration. The formulations may conveniently be presented in unit dosage form by methods known in the art of pharmacy.

The active ingredients of the invention may be formulated in similar or dissimilar pharmaceurical compositions and unit forms thereof.

If a solid carrier is used for oral administration, the preparation may be tablette, placed in a hard gelatine capsule in powder or pellet form or it may be in the form of a troche or lozenge.

The amount of solid carrier will vary widely but will usually be from about 25 mg to about 1 g.

If a liquid carrier is used, the preparation may be in the form of a syrup, emulsion, soft gelatine capsule or sterile injectable liquid such as an aqueous or non-aqueous liquid suspension or solution.

If desired, the pharmaceutical composition of the invention may comprise one or more active ingredients in combination with further pharmacologically active substances such as those described in the foregoing.

Materials and Methods

[³H]-Glycine Uptake

The GlyT-1 inhibitors for use in combination with an SRI, such as an SSRI, are tested in the well-recognised and reliable test measuring glycine uptake:

Cells transfected with the human GlyT-1b were seeded in 96 well plates. Prior to the experiment the cells were washed twice in HBS (10 mM Hepes-tris (pH 7,4), 2.5 mM KCl, 1 mM CaCl₂, 2.5 mM MgSO₄,) and pre-incubated with test compound for 6 minutes. Afterwards, 10 nM ³H-glycine was added to each well and the incubation was continued for 15 minutes. The cells were washed twice in HBS. Scintillation fluid was added and the Plates were counted on a Trilux (Wallac) scintillation counter.

Based on this test, compounds which are GlyT-1 inhibitors show inhibition below 20000 nM as IC₅₀ in the above-mentioned assay, preferably below 10000 nM.

Inhibition of the Uptake of [³H]Serotonin into Whole Rat Brain Synaptosomes

The inhibition of the serotonin uptake of an SRI is tested in the well-recognised and reliable test measuring serotonin uptake:

The compounds were tested with respect to their 5-HT reuptake inhibiting effect by measuring their ability to inhibit the uptake of [³H]serotonin into whole rat brain synaptosomes in vitro. The assay was performed as described by Hyttel Psychopharmacology 1978, 60, 13.

Based on this test, compounds which are SRI exhibit serotonin reuptake inhibition below 10000 nM (IC₅₀) in the assay above.

Animals

Male albino rats of a Wistar-derived strain (285-320 g; Harlan, Zeist, The Netherlands) were used for the experiments. Upon surgery, rats were housed individually in plastic cages (35×35×40 cm), and had free access to food and water. Animals were kept on a 12 h light schedule (light on 7:00 a.m.). The experiments are concordant with the declarations of Helsinki and were approved by the animal care committee of the faculty of mathematics and natural science of the University of Groningen.

Drugs

The following drugs were used: Citalopram hydrobromide and NFPS having the structure: 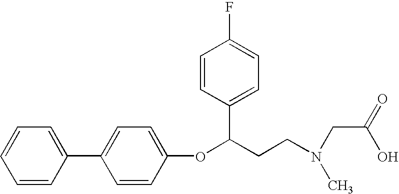
(LU 2736N) (Lundbeck A/S, Copenhagen, Denmark). Drugs were dissolved in saline and administered s.c.
Surgery

Microdialysis of brain serotonin levels was performed using home made I-shaped probes, made of polyacrylonitrile/sodium methyl sulfonate copolymer dialysis fiber (i.d. 220 μm, o.d. 0.31 μm, AN 69, Hospal, Italy). Preceding surgery rats were anaesthetised using isoflurane (O₂/N₂O; 300/300 ml/min). Lidocaine-HCl, 10% (m/v) was used for local anaesthesia. Rats were placed in a stereotaxic frame (Kopf, USA), and probes were inserted into Ventral Hippcampus (V. Hippo, L+4.8 mm, IA: +3.7 mm, V: −8.0 mm) (Paxinos and Watson, 1982). After insertion, probes were secured with dental cement.

Microdialysis Experiments

Rats were allowed to recover for at least 24 h. Probes were perfused with artificial cerebrospinal fluid containing 147 mM NaCl, 3.0 mM KCl, 1.2 mM CaCl₂, and 1.2 mM MgCl₂, at a flow-rate of 1.5 μl/min (Harvard apparatus, South Natick, Mass., USA). 15 minute microdialysis samples were collected in HPLC vials containing 7.5 μl 0.02 M acetic acid for serotonin analysis.

Serotonin Analysis:

Twenty-μl microdialysate samples were injected via an autoinjector (CMA/200 refrigerated microsampler, CMA, Sweden) onto a 100×2.0 mm C18 Hypersil 3 μm column (Bester, Amstelveen, the Netherlands) and separated with a mobile phase consisting of 5 g/L di-ammoniumsulfate, 500 mg/L EDTA, 50 mg/L heptane sulphonic acid, 4% methanol v/v, and 30 μl/L of triethylamine, pH 4.65 at a flow of 0.4 ml/min (Shimadzu LC-10 AD). 5-HT was detected amperometrically at a glassy carbon electrode at 500 mV vs Ag/AgCl (Antec Leyden, Leiden, The Netherlands). The detection limit was 0.5 fmol 5-HT per 20 μl sample (signal to noise ratio 3).

Data Presentation and Statistics

Four consecutive microdialysis samples with less then 20% variation were taken as control and set at 100%. Data are presented as percentages of control level (mean±S.E.M.) in time. Statistical analysis was performed using Sigmastat for Windows (SPSS, Jandel Corporation). Treatments were compared versus controls using two way analysis of variance (ANOVA) for repeated measurements, followed by Student Newman Keuls test. Drug effects were evaluated using one way ANOVA for repeated measures on ranks. Level of significance level was set at p<0.05.

Results

Co-Administration of Citalopram with NFPS on 5-HT Levels in Ventral Hippocampus

Administration of the glycine transporter inhibitor, NFPS (LU 2736N), at a dose of 10 μmol/kg s.c. did not induce any significant effects on serotonin levels in rat ventral hippocampus (X²₁₀=5.45 P=0.857). Co-administration of citalopram (10 μmol/kg s.c.) together with the Glycine transporter inhibitor NFPS (10 μmol/kg s.c.) significantly augmented the effect of citalopram on hippocampal serotonin levels (Treatment F(1,9)=5.35, P=0.044, Treatment vs. Time F(1,104)=2.12, P=0.033).

1. A method of treating a disorder selected from depression, anxiety disorders and other affective disorders, eating disorders phobias, dysthymia, premenstrual syndrome, cognitive disorders, impulse control disorders, attention deficit hyperactivity disorder, drug abuse or any other disorder responsive to serotonin reuptake inhibitors in a person in need thereof, the method comprising administering to the person a serotonin reuptake inhibitor and a compound which is a GlyT-1 inhibitor.

2. (canceled)

3. A method for augmenting and/or providing faster onset of the therapeutic effect of a serotonin reuptake inhibitor in a person comprising administering a serotonin reuptake inhibitor and a compound which is a GlyT-1 inhibitor to the person.

4. The method according to claim 3 wherein the person suffers from a disorder selected from depression, anxiety disorders and other affective disorders, eating disorders, phobias, dysthymia, premenstrual syndrome, cognitive disorders, impulse control disorders, attention deficit hyperactivity disorder, drug abuse or any other disorder responsive to a SRI.

5. The method of claim 1 wherein the SRI is a SSRI.

6. The method of claim 1 wherein the SRI is selected from citalopram, escitalopram, fluoxetine, sertraline, paroxetine, fluvoxamine, venlafaxine, dapoxetine, duloxetine, vilazodone, nefazodone, imipramin, femoxetine and clomipramine.

7. The method of claim 1 wherein the GlyT-1 inhibitor is selected from
N-{3-[5-Cyano-1-(4-fluorophenyl)-1,3-dihydroisobenzofuran-1-yl]-1-propyl}glycine ethyl ester,
N-{3-[5-Cyano-1-(4-fluorophenyl)-1,3-dihydroisobenzofuran-1-yl]-1-propyl}-N-methylglycine ethyl ester,
N-{3-[5-Cyano-1-(4-fluorophenyl)-1,3-dihydroisobenzofuran-1-yl]-1-propyl}glycine,
N-{3-[5-Cyano-1-(4-fluorophenyl)-1,3-dihydroisobenzofuran-1-yl]-1-propyl}-N-methylglycine,
N-{3-[1-(3-chlorophenyl)-1,3-dihydroisobenzofuran-1-yl]-1-propyl}-N-methylglycine,
N-{3-[1-(3-trifluoromethylphenyl)-1,3-dihydroisobenzofuran-1-yl]-1-propyl}-N-methylglycine,
N-{3-[1-(3-trifluoromethylphenyl)-1,3-dihydroisobenzofuran-1-yl]-1-propyl}-N-methyl(1-ethyl)glycine,
N-{3-[1-(4-methylphenyl)-1,3-dihydroisobenzofuran-1-yl]-1-propyl}-N-methylglycine,
N-{3-[1-(4-fluorophenyl)-1,3-dihydroisobenzofuran-1-yl]-1-propyl}-N-methylglycine,
N-{3-[1-(4-fluorophenyl)-1,3-dihydroisobenzofuran-1-yl]-1-propyl}-N-methylalanine,
N-{3-[1-(4-fluorophenyl)-1,3-dihydroisobenzofuran-1-yl]-1-propyl}-N-methyl(1-ethyl)glycine,
N-{3-[4-chloro-1-(3-methyl-4-fluorophenyl)-1,3-dihydroisobenzofuran-1-yl]-1-propyl}-N-methylglycine,
N-{3-[4-chloro-1-(4-chlorophenyl)-1,3-dihydroisobenzofuran-1-yl]-1-propyl}-N-methylglycine,
N-{3-[5-chloro-1-(4-chlorophenyl)-1,3-dihydroisobenzofuran-1-yl]-1-propyl}-N-methylalanine,
N-{3-[6-chloro-1-(3-methyl-4-fluorophenyl)-1,3-dihydroisobenzofuran-1-yl]-1-propyl}-N-methylglycine,
N-{3-[6-chloro-1-(4-chlorophenyl)-1,3-dihydroisobenzofuran-1-yl]-1-propyl}-N-methylglycine,
N-{3-[6-chloro-1-(4-methylphenyl)-1,3-dihydroisobenzofuran-1-yl]-1-propyl}-N-methylglycine,
N-{3-[6-chloro-1-(4-methoxyphenyl)-1,3-dihydroisobenzofuran-1-yl]-1-propyl}-N-methylglycine,
N-{3-[5-fluoro-1-(4-chlorophenyl)-1,3-dihydroisobenzofuran-1-yl]-1-propyl}-N-methylglycine,
N-{3-[5-fluoro-1-(4-methoxyphenyl)-1,3-dihydroisobenzofuran-1-yl]-1-propyl}-N-methylglycine,
N-{3-[5-trifluoromethyl-1-(4-fluorophenyl)-1,3-dihydroisobenzofuran-1-yl]-1-propyl}-N-methylglycine,
N-{3-[5-trifluoromethyl-1-(4-fluorophenyl)-1,3-dihydroisobenzofuran-1-yl]-1-propyl}-N-methylalanine,
N-{3-[5-cyano-1-(3-methyl-4-fluorophenyl)-1,3-dihydroisobenzofuran-1-yl]-1-propyl}-N-methylglycine,
N-{3-[5-cyano-1-(4-cyanophenyl)-1,3-dihydroisobenzofuran-1-yl]-1-propyl}-N-methylalanine,
N-{3-[5-cyano-1-(4-methoxyphenyl)-1,3-dihydroisobenzofuran-1-yl]-1-propyl}-N-methylglycine,
N-{3-[5-cyano-1-(4-fluorophenyl)-1,3-dihydroisobenzofuran-1-yl]-1-propyl}-N-methylglycine, N-{2-[5-cyano-1-(4-fluorophenyl)-1,3-dihydroisobenzofuran-1-yl]ethyl}-N-methylglycine,
N-{3-[5-Chloro-1-(4-chloro-phenyl)-indan-1-yl]-propyl}-N-methylglycine,
N-{3-[5-Chloro-1-(4-chloro-phenyl)-indan-1-yl]-propyl}-N-methylalanine,
N-{3-[3-cyclo-1-(4-methylphenyl)-1,3-dihydroisobenzofuran-1-yl]-1-propyl}-N-methylglycine,
N-[3-(3,3-Dimethyl-1-phenyl-1,3-dihydro-benzo[c]thiophen-1-yl)-propyl]-N-methylglycine,
N-[3-(3,3-Dimethyl-1-phenyl-1,3-dihydro-benzo[c]thiophen-1-yl)-propyl]-N-methylalanine,
N-{3-[1-(4-Fluoro-phenyl)-3,3-dimethyl-1,3-dihydro-isobenzofuran-1-yl]-propyl}-N-methylglycine,
N-{3-[5-Bromo-1-(4-chlorophenyl)-1,3-dihydroisobenzofuran-1-yl]-1-propyl}-N-methylglycine,
N-{2-1-(4-Chloro-phenyl)-3,3-dimethyl-1,3-dihydro-isobenzofuran-1-yl]-ethyl}-N-methylglycine,
N-[3-(3-methyl-1-phenyl-1H-inden-1-yl)-propyl]-N-methylglycine,
N-[3-(5-Chloro-1-thiophen-2-yl-1,3-dihydro-isobenzofuran-1-yl)-propyl]-N-methylglycine,
N-[3-(5-Chloro-1-thiophen-2-yl-1,3-dihydro-isobenzofuran-1-yl)-propyl]-N-methyl (1-ethyl)-glycine,
N-[3-(3-methyl-1-phenyl-1,3-dihydro-isobenzofuran-1-yl)-propyl]-N-methylalanine,
N-[3-(3-methyl-1-phenyl-1,3-dihydro-isobenzofuran-1-yl)-propyl]-N-methyl (1-ethyl)-glycine,
N-[3-(3,3-Dimethyl-1-phenyl-1,3-dihydro-isobenzofuran-1-yl)-ethyl]-N-methylalanine,
N-[3-(3,3-Dimethyl-1-(4-fluoro-phenyl)-1,3-dihydro-isobenzofuran-1-yl)-ethyl]-N-methylalanine,
N-[3-(3,3-Dimethyl-1-phenyl-1,3-dihydro-isobenzofuran-1-yl)-ethyl]-N-methyl-(1-ethyl)glycine,
N-[3-(3,3-Dimethyl-1-(4-fluoro-phenyl)-1,3-dihydro-isobenzofuran-1-yl)-ethyl]-N-methyl-(1-ethyl)glycine,
N-[3-(3,3-Diethyl-1-phenyl-1,3-dihydro-isobenzofuran-1-yl)-propyl]-N-methylalanine,
N-[3-(3,3-Diethyl-1-(4-chloro-phenyl)-1,3-dihydro-isobenzofuran-1-yl)-propyl]-N-methylalanine,
N-[3-(3,3-Diethyl-1-(4-chloro-phenyl)-1,3-dihydro-isobenzofuran-1-yl)-propyl]-N-methylglycine,
N-[3-(1-phenyl-1,3-dihydro-benzo[c]thiophen-1-yl)-propyl]-N-methylalanine,
N-{3-[1-(4-Chloro-phenyl)-3,3-dimethyl-indan-1-yl]-propyl}-N-methylglycine,
N-{3-[1-(4-Chloro-phenyl)-3,3-diethyl-1,3-dihydro-isobenzofuran-1-yl]-propyl}-N-methyl-alanine,
N-[2-(3-methyl-1-phenyl-indan-1-yl)-ethyl]-amino}-N-methyl alanine,
N-[3-(1-phenyl-(1H)-inden-1-yl)-propyl]-N-methyl-alanine,
N-{3-[1-(4-Fluoro-phenyl)-5-(4-trifluoromethyl-phenyl)-1,3-dihydro-isobenzofuran-1-yl]-propyl}-N-methyl-glycine,
N-{3-[5-Chloro-1-(4-chloro-phenyl)-indan-1-yl]-propyl}-N-methyl-glycine,
N-{3-[5-Chloro-1-(4-chloro-phenyl)-indan-1-yl]-propyl}-N-methyl-alanine,
N-{3-[1-(4-chloro-phenyl)-5-(4-trifluoromethyl-phenyl)-1,3-dihydro-isobenzofuran-1-yl]-ethyl}-N-methyl-glycine,
N-{3-[1-(4-Chloro-phenyl)-5-(4-methyl-phenyl)-1,3-dihydro-isobenzofuran-1-yl]-ethyl}-N-methyl-glycine,
N-{3-[1-(4-Chloro-phenyl)-5-(4-methoxy-phenyl)-1,3-dihydro-isobenzofuran-1-yl]-ethyl}-N-methyl-glycine,
N-{3-[1-(4-Chloro-phenyl)-5-(2-thiophenyl)-1,3-dihydro-isobenzofuran-1-yl]-ethyl}-N-methyl-glycine,
N-{3-[1-(4-Chloro-phenyl)-5-(4-methyl-phenyl)-1,3-dihydro-isobenzofuran-1-yl]-propyl}-N-methyl-glycine,
N-{3-[1-(4-Chloro-phenyl)-5-(4-methoxy-phenyl)-1,3-dihydro-isobenzofuran-1-yl]-propyl}-N-methyl-glycine,
N-{3-[-(4-chloro-phenyl)-5-(4-trifluoromethyl-phenyl)-1,3-dihydro-isobenzofuran-1-yl]-propyl}-N-methyl-glycine,
N-{3-[1-(4-Chloro-phenyl)-5-(4-chloro-phenyl)-1,3-dihydro-isobenzofuran-1-yl]-ethyl}-N-methyl-glycine,
N-{2-[1-(4-Chloro-phenyl)-5-(5-chloro-thiophen-2-yl)-1,3-dihydro-isobenzofuran-1-yl]-ethyl}-N-methyl-glycine,
N-{3-[1-(4-Chloro-phenyl)-5-(3-methyl-phenyl)-1,3-dihydro-isobenzofuran-1-yl]-ethyl}-N-methyl-glycine,
N-{3-[1-(4-Chloro-phenyl)-5-(2-methyl-phenyl)-1,3-dihydro-isobenzofuran-1-yl]-ethyl}-N-methyl-glycine,
N-{3-[1-(4-Chloro-phenyl)-5-(2,5-dichloro-phenyl)-1,3-dihydro-isobenzofuran-1-yl]-ethyl}-N-methyl-glycine,
N-{3-[1-(4-chloro-phenyl)-5-(3-trifluoromethyl-phenyl)-1,3-dihydro-isobenzofuran-1-yl]-ethyl}-N-methyl-glycine,
N-{3-[1-(4-chloro-phenyl)-5-(3-trifluoromethyl-phenyl)-1,3-dihydro-isobenzofuran-1-yl]-propyl}-N-methyl-glycine,
N-{3-[1-(4-Chloro-phenyl)-5-(3,4-dichloro-phenyl)-1,3-dihydro-isobenzofuran-1-yl]-ethyl}-N-methyl-glycine,
N-{3-[1-(4-Chloro-phenyl)-5-(4-chloro-phenyl)-1,3-dihydro-isobenzofuran-1-yl]-propyl}-N-methyl-glycine,
N-{3-[1-(4-Chloro-phenyl)-5-(3-methyl-phenyl)-1,3-dihydro-isobenzofuran-1-yl]-propyl}-N-methyl-glycine,
N-{3-[1-(4-Chloro-phenyl)-5-(2-methyl-phenyl)-1,3-dihydro-isobenzofuran-1-yl]-propyl}-N-methyl-glycine,
N-{3-[1-(4-Chloro-phenyl)-5-(2,5-dichloro-phenyl)-1,3-dihydro-isobenzofuran-1-yl]-propyl}-N-methyl-glycine,
N-{3-[1-(4-Chloro-phenyl)-5-(3,4-dichloro-phenyl)-1,3-dihydro-isobenzofuran-1-yl]-propyl}-N-methyl-glycine,
N-{3-[1-(4-chloro-phenyl)-5-(2-trifluoromethyl-phenyl)-1,3-dihydro-isobenzofuran-1-yl]-propyl}-N-methyl-glycine,
(±)-{4-[2-(4-Methoxy-phenylsulfanyl)-phenyl]-trans-2,5-dimethyl-piperazin-1-yl}-acetic acid,
(±)-{4-[2-(4-Chloro-phenylsulfanyl)-phenyl]-trans-2,5-dimethyl-piperazin-1-yl}-acetic acid,
(±)-{4-[2-(4-tert-Butyl-phenylsulfanyl)-phenyl]-trans-2,5-dimethyl-piperazin-1-yl}-acetic acid,
(±)-{4-[2-(4-Fluoro-phenylsulfanyl)-phenyl]-trans-2,5-dimethyl-piperazin-1-yl}-acetic acid,
(±)-{4-[2-(4-tert-Butyl-phenylsulfanyl)-phenyl]-2-methyl-piperazin-1-yl}-acetic acid,
(±)-{4-[2-(4-iso-Propyl-phenylsulfanyl)-phenyl]-2-methyl-piperazin-1-yl}-acetic acid,
(±)-2-{4-[2-(4-tert-Butyl-phenylsulfanyl)-phenyl]-trans-2,5-dimethylpiperazin-1-yl}-propionic acid,
{4-[5-Chloro-2-(4-methoxy-phenylsulfanyl)-phenyl]-2(R)-methyl-piperazin-1-yl}-acetic acid,
{4-[2-(4-Methoxy-phenylsulfanyl)-phenyl]-2(R),5(S)-dimethyl-piperazin-1-yl}-acetic acid,
{4-[5-Chloro-2-(4-methoxy-phenylsulfanyl)-phenyl]-2,2-dimethyl-piperazin-1-yl}-acetic acid,
(±)-{4-[5-Chloro-2-(4-trifluoromethyl-phenylsulfanyl)-phenyl]-2-methyl-piperazin-1-yl}-acetic acid,
{4-[5-Chloro-2-(3-methoxy-phenylsulfanyl)-phenyl]-2(R)-methyl-piperazin-1-yl}-acetic acid,
(±)-{4-[2-(4-Phenyl-phenyloxy)-phenyl]-2-methyl-piperazin-1-yl}-acetic acid,
(±)-{4-[2-(4-Methyl-phenylsulfanyl)-phenyl]-trans-2,5-dimethyl-piperazin-1-yl}-acetic acid,
(±)-{4-[2-(4-iso-Propyl-phenylsulfanyl)-phenyl]-trans-2,5-dimethyl-piperazin-1-yl}-acetic acid,
(±)-{4-[2-(2,4-Dimethyl-phenylsulfanyl)-phenyl]-trans-2,5-dimethyl-piperazin-1-yl}-acetic acid,
(±)-2-{4-[2-(4-tert-Butyl-phenylsulfanyl)-phenyl]-3-methylpiperazin-1-yl}-propionic acid,
{4-[2-(4-Isopropyl-phenylsulfanyl)-phenyl]-piperazin-1-yl}-acetic acid,
(±)-2-{4-[2-(4-Methoxy-phenylsulfanyl)-phenyl]-3-methyl-piperazin-1-yl}-propionic acid,
or a pharmaceutically acceptable acid addition salt thereof.

8. A pharmaceutical composition comprising a compound, which is a serotonin reuptake inhibitor, and a compound, which is a GlyT-1 inhibitor, and optionally a pharmaceutically acceptable carrier or diluent.

9. The pharmaceutical composition of claim 8 wherein the SRI is a SSRI.

10. The pharmaceutical composition of claim 8 wherein the GlyT-1 inhibitor shows inhibition below 20000 nM as IC₅₀ in the “[³H]-Glycine uptake” test.

11. The pharmaceutical composition of claim 8 wherein the GlyT-1 inhibitor is selected from
N-{3-[5-Cyano-1-(4-fluorophenyl)-1,3-dihydroisobenzofuran-1-yl]-1-propyl}glycine ethyl ester,
N-{3-[5-Cyano-1-(4-fluorophenyl)-1,3-dihydroisobenzofuran-1-yl]-1-propyl}-N-methylglycine ethyl ester,
N-{3-[5-Cyano-1-(4-fluorophenyl)-1,3-dihydroisobenzofuran-1-yl]-1-propyl}glycine,
N-{3-[5-Cyano-1-(4-fluorophenyl)-1,3-dihydroisobenzofuran-1-yl]-1-propyl}-N-methylglycine,
N-{3-[1-(3-chlorophenyl)-1,3-dihydroisobenzofuran-1-yl-1-propyl}-N-methylglycine,
N-{3-[1-(3-trifluoromethylphenyl)-1,3-dihydroisobenzofuran-1-yl]-1-propyl}-N-methylglycine,
N-{3-[1-(3-trifluoromethylphenyl)-1,3-dihydroisobenzofuran-1-yl]-1-propyl}-N-methyl(1-ethyl)glycine,
N-{3-[1-(4-methylphenyl)-1,3-dihydroisobenzofuran-1-yl]-1-propyl}-N-methylglycine,
N-{3-[1-(4-fluorophenyl)-1,3-dihydroisobenzofuran-1-yl]-1-propyl}-N-methylglycine,
N-{3-[1-(4-fluorophenyl)-1,3-dihydroisobenzofuran-1-yl]-1-propyl}-N-methylalanine,
N-{3-[1-(4-fluorophenyl)-1,3-dihydroisobenzofuran-1-yl]-1-propyl}-N-methyl(1-ethyl)glycine,
N-{3-[4-chloro-1-(3-methyl-4-fluorophenyl)-1,3-dihydroisobenzofuran-1-yl]-1-propyl}-N-methylglycine,
N-{3-[4-chloro-1-(4-chlorophenyl)-1,3-dihydroisobenzofuran-1-yl]-1-propyl}-N-methylglycine,
N-{3-[5-chloro-1-(4-chlorophenyl)-1,3-dihydroisobenzofuran-1-yl]-1-propyl}-N-methylalanine,
N-{3-[6-chloro-1-(3-methyl-4-fluorophenyl)-1,3-dihydroisobenzofuran-1-yl]-1-propyl}-N-methylglycine,
N-{3-[6-chloro-1-(4-chlorophenyl)-1,3-dihydroisobenzofuran-1-yl]-1-propyl}-N-methylglycine,
N-{3-[6-chloro-1-(4-methylphenyl)-1,3-dihydroisobenzofuran-1-yl]-1-propyl}-N-methylglycine,
N-{3-[6-chloro-1-(4-methoxyphenyl)-1,3-dihydroisobenzofuran-1-yl]-1-propyl}-N-methylglycine,
N-{3-[5-fluoro-1-(4-chlorophenyl)-1,3-dihydroisobenzofuran-1-yl]-1-propyl}-N-methylglycine,
N-{3-[5-fluoro-1-(4-methoxyphenyl)-1,3-dihydroisobenzofuran-1-yl]-1-propyl}-N-methylglycine,
N-{3-[5-trifluoromethyl-1-(4-fluorophenyl)-1,3-dihydroisobenzofuran-1-yl]-1-propyl}-N-methylglycine,
N-{3-[5-trifluoromethyl-1-(4-fluorophenyl)-1,3-dihydroisobenzofuran-1-yl]-1-propyl}-N-methylalanine.
N-{3-[5-cyano-1-(3-methyl-4-fluorophenyl)-1,3-dihydroisobenzofuran-1-yl-1-propyl}-N-methylglycine,
N-{3-[5-cyano-1-(4-cyanophenyl)-1,3-dihydroisobenzofuran-1-yl]-1-propyl}-N-methylalanine,
N-{3-[5-cyano-1-(4-methoxyphenyl)-1,3-dihydroisobenzofuran-1-yl]-1-propyl}-N-methylglycine,
N-{3-[5-cyano-1-(4-fluorophenyl)-1,3-dihydroisobenzofuran-1-yl]-1-propyl}-N-methylglycine, N-{2-[5-cyano-1-(4-fluorophenyl)-1,3-dihydroisobenzofuran-1-yl]ethyl}-N-methylglycine,
N-{3-[5-Chloro-1-(4-chloro-phenyl)-indan-1-yl]-propyl]-N-methylglycine,
N-{3-[5-Chloro-1-(4-chloro-phenyl)-indan-1-yl]-propyl]-N-methylalanine,
N-{3-[3-cyclo-1-(4-methylphenyl)-1,3-dihydroisobenzofuran-1-yl]-1-propyl}-N-methylglycine.
N-[3-(3,3-Dimethyl-1-phenyl-1,3-dihydro-benzo[c]thiophen-1-yl)-propyl]-N-methylglycine,
N-[3-(3,3-Dimethyl-1-phenyl-1,3-dihydro-benzo[c]thiophen-1-yl)-propyl]-N-methylalanine,
N-{3-[1-(4-Fluoro-phenyl)-3,3-dimethyl-1,3-dihydro-isobenzofuran-1-yl]-propyl}-N-methylglycine,
N-{3-[5-Bromo-1-(4-chlorophenyl)-1,3-dihydroisobenzofuran-1-yl]-1-propyl}-N-methylglycine,
N-{2-[1-(4-Chloro-phenyl)-3,3-dimethyl-1,3-dihydro-isobenzofuran-1-yl]-ethyl}-N-methylglycine.
N-[3-(3-methyl-1-phenyl-1H-inden-1-yl)-propyl]-N-methylglycine,
N-[3-(5-Chloro-1-thiophen-2-yl-1,3-dihydro-isobenzofuran-1-yl)-propyl}-N-methylglycine,
N-[3-(5-Chloro-1-thiophen-2-yl-1,3-dihydro-isobenzofuran-1-yl)-propyl}-N-methyl(1-ethyl)-glycine,
N-[3-(3-methyl-1-phenyl-1,3-dihydro-isobenzofuran-1-yl)-propyl]-N-methylalanine,
N-[3-(3-methyl-1-phenyl-1,3-dihydro-isobenzofuran-1-yl)-propyl]-N-methyl(1-ethyl)-glycine,
N-[3-(3,3-Dimethyl-1-phenyl-1,3-dihydro-isobenzofuran-1-yl)-ethyl]-N-methylalanine,
N-[3-(3,3-Dimethyl-1-(4-fluoro-phenyl)-1,3-dihydro-isobenzofuran-1-yl)-ethyl]-N-methylalanine,
N-[3-(3,3-Dimethyl-1-phenyl-1,3-dihydro-isobenzofuran-1-yl)-ethyl]-N-methyl-(1-ethyl)glycine,
N-[3-(3,3-Dimethyl-1-(4-fluoro-phenyl)-1,3-dihydro-isobenzofuran-1-yl)-ethyl]-N-methyl-(1-ethyl)glycine,
N-[3-(3,3-Diethyl-1-phenyl-1,3-dihydro-isobenzofuran-1-yl)-propyl]-N-methylalanine,
N-[3-(3,3-Diethyl-1-(4-chloro-phenyl)-1,3-dihydro-isobenzofuran-1-yl)-propyl]-N-methylalanine,
N-[3-(3,3-Diethyl-1-(4-chloro-phenyl)-1,3-dihydro-isobenzofuran-1-yl)-propyl]-N-methylglycine,
N-[3-(1-phenyl-1,3-dihydro-benzo[c]thiophen-1-yl)-propyl]-N-methylalanine,
N-{3-[1-(4-Chloro-phenyl)-3,3-dimethyl-indan-1-yl]-propyl}-N-methylglycine,
N-{3-[1-(4-Chloro-phenyl)-3,3-diethyl-1,3-dihydro-isobenzofuran-1-yl]-propyl}-N-methyl-alanine,
N-[2-(3-methyl-1-phenyl-indan-1-yl)-ethyl]-amino}-N-methyl alanine,
N-[3-(1-phenyl-(1H)-inden-1-yl)-propyl]-N-methyl-alanine,
N-{3-[1-(4-Fluoro-phenyl)-5-(4-trifluoromethyl-phenyl)-1,3-dihydro-isobenzofuran-1-yl]-propyl}-N-methyl-glycine,
N-{3-[5-Chloro-1-(4-chloro-phenyl)-indan-1-yl]-propyl}-N-methyl-glycine.
N-{3-[5-Chloro-1-(4-chloro-phenyl)-indan-1-yl]-propyl}-N-methyl-alanine,
N-{3-[1-(4-chloro-phenyl)-5-(4-trifluoromethyl-phenyl)-1,3-dihydro-isobenzofuran-1-yl]-ethyl}-N-methyl-glycine,
N-{3-[1-(4-Chloro-phenyl)-5-(4-methyl-phenyl)-1,3-dihydro-isobenzofuran-1-yl]-ethyl}-N-methyl-glycine.
N-{3-[1-(4-Chloro-phenyl)-5-(4-methoxy-phenyl)-1,3-dihydro-isobenzofuran-1-yl]-ethyl}-N-methyl-glycine,
N-{3-[1-(4-Chloro-phenyl)-5-(2-thiophenyl)-1,3-dihydro-isobenzofuran-1-yl]-ethyl}-N-methyl-glycine,
N-{3-[1-(4-Chloro-phenyl)-5-(4-methyl-phenyl)-1,3-dihydro-isobenzofuran-1-yl]-propyl}-N-methyl-glycine,
N-{3-[1-(4-Chloro-phenyl)-5-(4-methoxy-phenyl)-1,3-dihydro-isobenzofuran-1-yl]-propyl}-N-methyl-glycine,
N-{3-[1-(4-chloro-phenyl)-5-(4-trifluoromethyl-phenyl)-1,3-dihydro-isobenzofuran-1-yl]-propyl}-N-methyl-glycine,
N-{3-[1-(4-Chloro-phenyl)-5-(4-chloro-phenyl)-1,3-dihydro-isobenzofuran-1-yl]-ethyl}-N-methyl-glycine,
N-{2-[1-(4-Chloro-phenyl)-5-(5-chloro-thiophen-2-yl)-1,3-dihydro-isobenzofuran-1-yl]-ethyl}-N-methyl-glycine,
N-{3-[1-(4-Chloro-phenyl)-5-(3-methyl-phenyl)-1,3-dihydro-isobenzofuran-1-yl]-ethyl}-N-methyl-glycine,
N-{3-[1-(4-Chloro-phenyl)-5-(2-methyl-phenyl)-1,3-dihydro-isobenzofuran-1-yl]-ethyl}-N-methyl-glycine,
N-{3-[1-(4-Chloro-phenyl)-5-(2,5-dichloro-phenyl)-1,3-dihydro-isobenzofuran-1-yl]-ethyl}-N-methyl-glycine,
N-{3-[1-(4-chloro-phenyl)-5-(3-trifluoromethyl-phenyl)-1,3-dihydro-isobenzofuran-1-yl]-ethyl}-N-methyl-glycine,
N-{3-[1-(4-chloro-phenyl)-5-(3-trifluoromethyl-phenyl)-1,3-dihydro-isobenzofuran-1-yl]-propyl}-N-methyl-glycine
N-{3-[1-(4-Chloro-phenyl)-5-(3,4-dichloro-phenyl)-1,3-dihydro-isobenzofuran-1-yl]-ethyl}-N-methyl-glycine,
N-{3-[1-(4-Chloro-phenyl)-5-(4-chloro-phenyl)-1,3-dihydro-isobenzofuran-1-yl]-propyl}-N-methyl-glycine,
N-{3-[1-(4-Chloro-phenyl)-5-(3-methyl-phenyl)-1,3-dihydro-isobenzofuran-1-yl]-propyl}-N-methyl-glycine,
N-{3-[1-(4-Chloro-phenyl)-5-(2-methyl-phenyl)-1,3-dihydro-isobenzofuran-1-yl]-propyl}-N-methyl-glycine,
N-{3-[1-(4-Chloro-phenyl)-5-(2,5-dichloro-phenyl)-1,3-dihydro-isobenzofuran-1-yl]-propyl}-N-methyl-glycine,
N-{3-[1-(4-Chloro-phenyl)-5-(3,4-dichloro-phenyl)-1,3-dihydro-isobenzofuran-1-yl]-propyl}-N-methyl-glycine,
N-{3-[1-(4-chloro-phenyl)-5-(2-trifluoromethyl-phenyl)-1,3-dihydro-isobenzofuran-1-yl]-propyl}-N-methyl-glycine,
(±)-{4-[2-(4-Methoxy-phenylsulfanyl)-phenyl]-trans-2,5-dimethyl-piperazin-1-yl}-acetic acid,
(±)-{4-[2-(4-Chloro-phenylsulfanyl)-phenyl]-trans-2,5-dimethyl-piperazin-1-yl}-acetic acid,
(±)-{4-[2-(4-tert-Butyl-phenylsulfanyl)-phenyl-trans-2,5-dimethyl-piperazin-1-yl}-acetic acid,
(±)-{4-[2-(4-Fluoro-phenylsulfanyl)-phenyl]-trans-2,5-dimethyl-piperazin-1-yl}-acetic acid,
(±)-{4-[2-(4-tert-Butyl-phenylsulfanyl)-phenyl]-2-methyl-piperazin-1-yl}-acetic acid,
(±)-{4-[2-(4-iso-Propyl-phenylsulfanyl)-phenyl]-2-methyl-piperazin-1-yl}-acetic acid,
(±)-{2-{4-[2-(4-tert-Butyl-phenylsulfanyl)-phenyl]-trans-2,5-dimethylpiperazin-1-yl}-propionic acid,
{4-[5-Chloro-2-(4-methoxy-phenylsulfanyl)-phenyl]-2(R)-methyl-piperazin-1-yl}-acetic acid,
{4-[2-(4-Methoxy-phenylsulfanyl)-phenyl]-2(R),5(S)-dimethyl-piperazin-1-yl}-acetic acid,
{4-[5-Chloro-2-(4-methoxy-phenylsulfanyl)-phenyl]-2,2-dimethyl-piperazin-1-yl}-acetic acid,
(±)-{4-[5-Chloro-2-(4-trifluoromethyl-phenylsulfanyl)-phenyl]-2-methyl-piperazin-1-yl}-acetic acid,
{4-[5-Chloro-2-(3-methoxy-phenylsulfanyl)-phenyl]-2(R)-methyl-piperazin-1-yl}-acetic acid,
(±)-{4-[2-(4-Phenyl-phenyloxy)-phenyl]-2-methyl-piperazin-1-v}-acetic acid,
(±)-{4-[2-(4-Methyl-phenylsulfanyl)-phenyl]-trans-2,5-dimethyl-piperazin-1-yl}-acetic acid,
(±)-{4-[2-(4-iso-Propyl-phenylsulfanyl)-phenyl]-trans-2,5-dimethyl-piperazin-1-yl}-acetic acid,
(±)-{4-[2-(2,4-Dimethyl-phenylsulfanyl)-phenyl]-trans-2,5-dimethyl-piperazin-1-yl}-acetic acid,
(±)-2-{4-[2-(4-tert-Butyl-phenylsulfanyl)-phenyl]-3-methylpiperazin-1-yl}-propionic acid,
{4-[2-(4-Isopropyl-phenylsulfanyl)-phenyl]-piperazin-1-yl}-acetic acid,
(±)-2-{4-[2-(4-Methoxy-phenylsulfanyl)-phenyl]-3-methyl-piperazin-1-yl}-propionic acid,
or a pharmaceutically acceptable acid addition salt thereof.

12. The pharmaceutical composition according to claim 8 wherein the serotonin uptake inhibitor is selected from citalopram, escitalopram, fluoxetine, sertraline, paroxetine, fluvoxamine, venlafaxine, dapoxetine, duloxetine, vilazodone, nefazodone, imipramin, femoxetine and clomipramine.

13. The pharmaceutical composition according to claim 8, which is adapted for simultaneous administration of the active ingredients.

14. The pharmaceutical composition according to claim 13 wherein the active ingredients are contained in the same unit dosage form.

15. The pharmaceutical composition according to claim 8 which is adapted for sequential administration of the active ingredients.

16. The pharmaceutical composition according to claim 8 wherein the active ingredients are contained in discrete dosage forms.

17. A kit comprising a compound, which is a serotonin reuptake inhibitor, and a compound, which is a GlyT-1 inhibitor, and optionally a pharmaceutically acceptable carrier or diluent.

18. (canceled)

19. The pharmaceutical composition of claim 10, wherein the GlyT-1 inhibitor shows inhibition below 10000 nM, as IC₅₀ in the “[³H]-Glycine uptake” test.

20. The pharmaceutical composition according to claim 15, wherein the active ingredients are contained in discrete dosage forms.
